(19) United States
(12) Patent Application Publication
(10) Pub. No.: US 2003/0078477 A1
(43) Pub. Date: Apr. 24, 2003

(54) Fluorescence endoscope apparatus and method for imaging tissue within a body using the same

(75) Inventors: Uk Kang, Goonpo-si (KR); Garry V. Papayan, St. Petersburg (RU)

Correspondence Address: ARENT FOX KINTNER PLOTKIN & KAHN, PLLC, Suite 400, 1050 Connecticut Avenue, N.W., Washington, DC, 20036-5339, US

(73) Assignee: KOREA ELECTROTECHNOLOGY RESEARCH INSTITUTE

(21) Appl. No.: 10/266,592

(22) Filed: Oct. 9, 2002

(30) Foreign Application Priority Data
Oct. 18, 2001 (KR) 01-64441

(51) Int. Cl. A61B 1/06; A61B 10/00

(52) U.S. Cl. 600/178; 600/562; 600/109

(57) ABSTRACT

This present invention relates to a fluorescence endoscope apparatus, developed for diagnosing various illnesses within a body, especially for diagnosing a tumor inflamed region; and an application method of the same. The purpose of this invention is to enhance the accuracy of the examination. The fluorescence light endoscope apparatus in accordance with the present invention is comprised of an endoscope probe; a multiple light source that provides illumination light or excitation light of short wavelength onto a diagnostic region; a color CCD camera and a high sensitive monochromatic CCD camera placed on the back of an endoscope ocular lens; a reference test sample; a computer; and a monitor. The method of the present invention embodies a preliminary correction of the fluorescence endoscope apparatus of the present invention in accordance with the reference test sample; a general endoscopy using the illumination light, and an image observation and examination of the same diagnostic region using the fluorescence light and the reflected excitation light simultaneously; an auto-correction of brightness and unevenness of the fluorescence light images of the diagnostic region according to the reference test sample data; an evaluation of the brightness of the fluorescence light in the diagnosis region; storing numerical data that characterizing images and brightness of the fluorescence light in the diagnosis region; and storing image collected via two cameras as digital video clips.

### BACKGROUND OF THE INVENTION

1. Field of the Invention

The present invention relates to a fluorescence endoscope apparatus and a method for imaging tissue within a body cavity using the same, and in particular to a fluorescence endoscope apparatus and a method for examining tumor within a body.

2. Description of the Related Art

In general, existing general endoscopes are categorized into a fiber optical endoscope, which uses optical fibers and an optical lens; and an electron microscope that uses optical fibers for projecting light and has a receiver that converts image signals into electronic signals through a CCD micro chip mounted at the distal end of an endoscope probe which can be observed through a monitor. On the other hand, the fiber optical endoscope can also use a monitor for an observation, in this case the observation through a monitor can be done after converting image signals into electronic signals through a CCD camera installed on the back of an ocular lens of the same.

Therefore, by using the conventional endoscope system, a user can see the diagnostic part of an internal organ, such as a stomach, through either a monitor using a color CCD camera, or observing directly with the naked eyes through an endoscope comprised of a bundle of optical fibers.

In the case of the conventional fluorescence endoscope system, an illumination light source is used for viewing the internal organs, just as it's done with the existing general endoscopes. Moreover, the fluorescence endoscope system is further comprised of an excitation light source for observing either the difference in autofluorescence light intensity within a body tissue according to an existence or a nonexistence of inflamed body tissues of the internal organs; or the difference in secondary fluorescence light intensity between the inflamed region and the normal region, after injecting a contrast agent into a body. A user can use such an endoscope system to either observe a specific region in the interior of a body just as it is done with the general endoscopes, or in the case where a suspected diseased region was found, the endoscope system can be used to easily identify and examine diseases, such as a malignant tumor, at an early stage by observing the difference in fluorescence light intensity of a suspicious region after converting into excitation light. However, when the fluorescence images are used to perform an examination, the excitation light source and the CCD camera need to be connected to the endoscope, and when using illumination light to perform an observation, the excitation light source and the CCD camera need to be detached from the endoscope. Such examination process has problems of expanding examination time of a patient, and a doctor, as a user, loses a chance to compare images obtained from fluorescence light and reflective light on the same region, which in turn causes an effectiveness reduction.

To solve such problems of the conventional fluorescence endoscopes, the U.S. Pat. No. 4,821,117 (1989) was proposed. This U.S. Patent has a description about a fluorescence endoscope system that enables a monitor to display two images at the same time by memorizing each image with a help of a computer buffer storing device after methodically collecting images, obtained from reflected illumination light and from fluorescence light produced by the excitation light, through a CCD camera.

However, it was known that the fluorescence endoscope system introduced in the U.S. Pat. No. 4,821,117 does not provide high definition video images. In other words, the television system requires different requirements in order to optimize endoscope images of the illumination light and the fluorescence light, where the illumination light requires high definition color television system, and the fluorescence light does not. However, since the required high sensitivity level is to be reached in monochrome television systems due to amplification of image intensifiers or use of a mode of signal accumulation the fluorescence endoscope of the U.S. Pat. No. 4,821,117, which processes images received from the reflected illumination light and the fluorescence light produced by the excitation light through a television camera, has a problem of not providing high definition video images. Also, the cyclic filming process for an image recording uses unnecessary fluorescence light energy during non-examining hours because of the excitation light. Moreover, the system is complicate and is big in size due to rotating optical instrument parts, which are big drawbacks.

More improved fluorescence endoscope than the previously described technology was proposed in the U.S. Pat. No. 5,827,190, the fluorescence endoscope system equipped with two CCD cameras. Analysis of images from the autofluorescence light was done in the U.S. Pat. No. 5,827,190, for the examination of malignant diseases, and also introduced the application method and the device. The summarized descriptions are as follow.

A light source, which projects light via an endoscope bundle, lights a diagnostic region with light sources of two different wavelengths; blue light as the excitation light to induce tissue autofluorescence and red/near-infrared light as the reflective light (backscattering light). Images of an object, collected from the fluorescence light and the reflective light, were projected simultaneously into two CCD cameras mounted at the distal end of an endoscope probe through the endoscope's object lens. Light splitting was done by dichroic mirrors that are fixed in front of the CCD cameras. This patent proposed a use of the images obtained from the reflected red light to compensate changes in fluorescence intensity due to the variation of distance and angle between the objective lens and the surface of the diagnostic region, and also proposed a use of blue excitation light and red illumination light when diagnosing a inflamed tissue that is turned red due to an infection. Since, however, there exists no illumination light source in the fluorescence endoscope system of the U.S. Pat. No. 5,827,190, general endoscope images cannot be observed with the illumination light that is important for a doctor, who performs the fluorescence endoscopy, to make a proper diagnosis. Moreover, the correction of the fluorescence image based on use of reference image in reflective light, cannot be accurate due to existing differences between the absorbed and the scattered radiations, and also inevitable bright spots in reflective light with different brightness distribution in comparison with fluorescence light. In other words, since redness varies along different body tissues, a doctor's job gets complicated in correct selection of wavelengths of backscattering light. Also, there exists a problem of accuracy degradation of diagnosis due to non-existence of measuring system for an estimation of brightness of the received fluorescence images.

### SUMMARY OF THE INVENTION

The present invention is invented to solve the existing problems described above. It is an object of the present invention to provide a fluorescence endoscope apparatus, which is designed to increase the accuracy of the fluorescence-endoscopy diagnostics by way, a possibility of observation of object in white light, an objective evaluation of fluorescence light intensity and decrease of its dependence on destabilizing factors; and a method for imaging tissue within a body using the same.

### BRIEF DESCRIPTION OF THE DRAWINGS

The accompanying drawings, which are included to provide further understandings of the invention, illustrate the preferred embodiments of the invention, and together with the description, serve to explain the principles of the present invention.

FIG. 1 is a block diagram illustrating a fluorescence endoscope apparatus in accordance with an exemplary embodiment of the present invention;

FIG. 2 is a flowchart illustrating a method for imaging tissue within a body in accordance with an exemplary embodiment of the apparatus of FIG. 1 of the present invention; and

FIG. 3 is a flowchart illustrating a reference test sample based endoscopy process in accordance with the present invention.

### DETAILED DESCRIPTION OF THE PREFFERRED EMBODIMENT

In order to facilitate an understanding of this invention, a fluorescence endoscope apparatus, an apparatus for examining tissue within a body, is comprised of, a multiple light source unit, equipped with multiple light sources of different wavelengths, to provide a selected light; a light transmission unit with an objective lens installed on the incidence path to make an insertion into a body possible, where the exit path, for transmitting and radiating the provided light, and the incidence path, for transmitting the incidence light in correspondence with the radiation, are formed in parallel; an light splitting unit for splitting the light transmitted via the incidence path into primary light and secondary light; a primary image processing unit for collecting primary images based on the primary light that has passed through; a secondary image processing unit for collecting secondary images based on the secondary light that has been reflected; a control unit for processing, analyzing, storing and synthesizing the collected primary and the secondary images; and a displaying unit for displaying the primary and the secondary images, which are processed through the control unit, or their synthesized images on to a screen.

The multiple-light source unit is designed to be equipped with illumination light and excitation light at least as the light sources; however, the light sources of the illumination light and the excitation light can be formed into two separate lamps or into a combined lamp in accordance with an output of light and a size of wavelength of the corresponding light source.

The light splitting unit is designed for excitation light to pass through or to be reflected according to the size of light wavelength; however, the unit should not be placed in a fixed position on the light path of the incidence light by a mechanical installation, in order to enable the unit to escape from the corresponding light path especially by the user selection.

Make the primary image processing unit to collect color images; the secondary image processing unit to collect high sensitive monochromatic images; and place objective lenses on the incidence paths of the primary and the secondary image processing units respectively. Moreover, install a light-shielding filter, which permeates only the fluorescence light from the incidence path of the secondary image-processing unit.

The control unit has characteristics of storing reference image data regarding the primary and/or the secondary images (especially the secondary image) of the corresponding object, in other words, storing the data regarding the standardized image of the corresponding object as the reference image. Then correct the image, which is collected as the secondary image, based on the reference image; however, the reference image is the image that is collected as the secondary image from the reference test sample of a model, which has the same or the similar optical characteristic compared to those of the actual object under examination.

The apparatus of the present invention, organized as above, comprises a multiple light source apparatus as the multiple light sources connected to a fiber optic cable of an endoscope. A lighting device, as the multiple light source device, is made by using light sources that are non-coherent. The lighting device provides either illumination light for a general observation, or excitation light of a short wavelength for a fluorescence examination onto a diagnostic region. By an optical system, images of a diagnostic region are transferred from the distal end of an endoscope probe to an ocular lens that is placed on the back of the endoscope. On the back of the ocular lens, a foldable dichroic mirror is placed as a light splitting unit, and a remote control switch is also placed on the back of the ocular lens as an optical switch that can interchange light sources of the lighting device. During an operation, light from the endoscope gets divided and transferred into the paths where two CCD cameras are placed, by the dichroic mirror. The CCD camera placed on the first path is in color. This camera is installed to collect reflective light images. The CCD camera placed on the second path is a high sensitive monochromic (black and white) camera, which is installed to collect fluorescence images. An objective lens is placed on the each path of the two CCD cameras. In addition, a light shielding filter, which permeates only the fluorescence radiation into the high sensitive CCD camera placed on the second path, is installed. Signals from the two CCD cameras are delivered to a computer that is used as the control unit. The computer controls the operation of the CCD system, and processes and analyzes images collected from the cameras. Main functions of the computer are; to correct and to measure accurate intensity of fluorescence images in real time; to display images collected from the two CCD cameras simultaneously in a dual mode or to display the synthesized images on a single monitor; and to store each image in the form of a separate or synthesized video clips. Besides, a reference test sample is included in the apparatus of the present invention, and the surface of this reference test sample is the same in all regions, and is designed to have an optical characteristic similar to that of the object under an inspection.

A preliminary correction of the system is performed by the reference test sample. The correction is done by storing fluorescence images of the reference test sample into the computer, in a fixed state. The stored data is used to correct unevenness of fluorescence images, which is caused by the difference in light accumulation on the diagnostic region due to unevenness of lighting as well as a field view of the endoscope. In addition, a lamp exchange and sensitivity correction of the device with an old lamp, are corrected according to the acquired data

In order to facilitate an understanding of this invention, a method for imaging tissue within a body using the fluorescence endoscope apparatus of the present invention is comprised of, collecting reference data, regarding standardized fluorescence images of a corresponding object under examination, from the reference test sample of a model that has optical characteristics that are same or similar to those of the object under examination; lighting the diagnostic part, as the object under examination, with illumination light; collecting and displaying color images, which are produced based on reflective light that is reflected by the lighting of the illumination light; lighting the diagnostic part with excited light that has a wide spectrum range; collecting high sensitive monochromatic fluorescence light images while collecting color excitation light images simultaneously; correcting the high sensitive monochromatic images and the brightness of the fluorescence light, which are obtained according to the collected reference data; and displaying the obtained high sensitive monochromatic image and the color image simultaneously in a dual mode, or displaying a single synthesized image of the two images on a screen.

The reflective light, which is generated from the diagnostic region by illumination light and excitation light, implies normal reflective light and backscattering light.

The method for imaging tissue within a body using the fluorescence endoscope apparatus of the present invention has characteristics of, mutually synthesizing the obtained monochromatic images and color images that are stored in the form of a digital video clip; calculating fluorescence light intensity of the monochromatic image through analyzing a histogram of relative image signal distribution regarding the displaying screen; and representing the calculated data of fluorescence light intensity along with the synthesized images, with digital numbers.

The method of the present invention, organized as above, starts a general endoscopy using the color CCD camera with illumination light lighting. A color screen, obtained from the general endoscopy, allows an observation for configuration and functional characteristics of the diagnostic region, and detection for a tissue site that is likely to have a tumor. Then follows a fluorescence endoscopy. The fluorescence image of the diagnostic region and reflected excitation light with a short wavelength will be displayed on the monitor screen. In the case of the latter fluorescence endoscopy, a user (normally a doctor) can easily locate the exact location of an examining organ with the endoscope, and can also easily control the location of the distal end of the endoscope probe since this endoscopy is performed in the spectrum of a wide range, between 380 nm and 580 nm. In the monochromatic CCD camera, an intensity of a detailed fluorescence image of the given brightness is evaluated according to an analysis of a histogram distribution of received display signals. In order to eliminate the measuring errors, which are generated due to the change in distance from the distal end of the endoscope to the surface of a diagnostic region, the distance is adjusted by inserting the endoscope instrument in from the insertion point to a specified value. Numerical data that characterizes the intensity of fluorescence light is to be displayed on the screen along with an image. Moreover, fixed conditions are also displayed along with an image, and the images collected from the two CCD cameras are stored into the computer in the form of digital video clips.

In order that the invention may be fully understood, a preferred embodiment thereof will now be described with reference to the accompanying drawings.

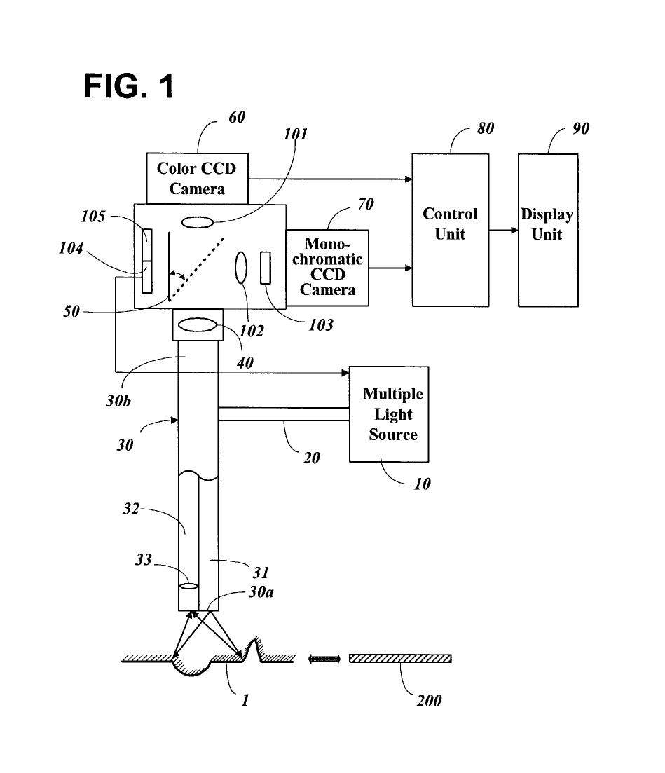

FIG. 1 is a block diagram illustrating a fluorescence endoscope apparatus in accordance with an exemplary embodiment of the present invention, which is comprised of, a multiple light source 10 that supplies illumination light and excitation light of a short wavelength in accordance with the selection; an optical cable 20 for transmitting light provided from the multiple light source 10. The apparatus is further provided with a flexible or a rigid endoscope probe 30, that can be defined into two parts: a distal end 30a, that is to be inserted into a body to observe the diagnostic region, and a proximal end 30b, that is to be located outside of a body. The endoscope probe 30 is also organized with a primary optical fiber bundle 31 for transmitting and radiating the transmitted light, from the optical cable 20, to provide lighting; a secondary fiber optic bundle 32 for transmitting incidence light that corresponds to the radiation, and is combined with the primary fiber optic bundle 31 in parallel; an objective lens 33, which located at the inserting end of the secondary fiber optic bundle 32; and an ocular lens 40, which is located at the end of the proximal end 30b. A dichroic mirror 50 is installed in the apparatus to inject the light, which is transmitted via the secondary fiber optic bundle 32 and the ocular lens, and it separates the incidence light into two kinds of light for two paths by letting through or reflecting the light according to its type (in other words, size of wavelengths). The dichroic mirror 50 is made foldable, especially in a mechanical point of view, to allow the device 50 to be placed selectively in the incidence paths. The fluorescence endoscope apparatus further comprises of, a color CCD camera 60, which inputs the light that passed through the dichroic mirror 50 and produces color images based on the same light; a high sensitive monochromatic CCD camera 70, which inputs the reflective light that is reflected from the dichroic mirror 50 and produces high sensitive monochromic images (or pictures) based on the same light; a controller 80, such as a computer, for inputting color or monochromatic images produced via the CCD cameras 60, 70, and for digital processing, analyzing, digital storing, and synthesizing the inputted images; a displaying device 90, such as a monitor, that displays the processed color images and the processed monochromatic images from the controller 80, or their synthesized images; objective lenses 101, 102 mounted on the light injecting front of the CCD cameras 60, 70; a light shielding filter 103, placed in between the objective lens 102 and the light-inputting section of the monochromatic CCD camera 70, to permeate light of specific wavelengths only; a light source switch 104 that is for selecting a light source type of the multiple light source 10 from a long distance; and a light path switch 105 that changes the path of the light, of which passes through the ocular lens 40, by setting the dichroic mirror 50 to be either folded or unfolded.

The light source switch 104 and the light path switch 105 are simultaneously controlled in mutual relation, according to the illumination light observation and the fluorescence light examination conditions. In other words, during the fluorescence endoscopy, the light source switch 104 makes the multiple light source 10 to meet the excitation light conditions, and at the same time, the light path switch 104 separates the fluorescence light and the reflective light from the excitation light by unfolding the dichroic mirror 50. Also, during the illumination light observation, the light source switch 104 makes the multiple light source 10 to meet the illumination light conditions, and at the same time, the light path switch 105 makes the dichroic mirror 50 not to be included in the light path by folding the dichroic mirror 50.

Moreover, in the present invention, a reference test sample 200 of a model that has the same or similar optical characteristics as the examining object (for instance, the stomach and bowels) is to be produced. Moreover, data regarding the standard images of a corresponding examining object is to be collected by using the apparatus of the present invention that is organized as shown in FIG. 1, in accordance with the reference test sample 200. Then the collected data is to be stored into the controller 80 as the reference data.

When organizing the multiple light source 10, non-coherent light must be used as the light source. For instance, organizing the multiple light source 10 with a halogen and a mercury lamp, as the light sources of the illumination light and the excitation light. For another example, the multiple light source 10 can be organized with a xenon lamp as the light source for both the illumination light and the excitation light.

In other words, in the examples, light of wide spectrum range of 380 nm-580 nm (this light is seen as blue-green light to human eyes, and scientifically the light is purple-green light) is to be used as the excitation light, and a contrast agent, which can allow fluorescence light from the excitation light to have a larger wavelength than 600 nm, is to be used. Therefore, a mercury lamp, which generates illumination of the spectrum range, can be used as a single light source, or a xenon lamp, which generates illumination of a range that includes both the wavelength range of illumination light and the spectrum range of 380 nm-580 nm, can be used as an integration lamp. In order to correspond with the organization of such multiple light source 10, the dichroic mirror 50 lets light with a wavelength range of 380 nm-580 nm to pass through, and reflects the light with a wavelength greater than 580 nm. The light shielding filter 103 is to absorb all lights of a wavelength shorter than 600 nm, then is to shield the absorbed lights.

Descriptions of components of the actual embodiment of the present invention, organized as in FIG. 1, are as follow.

For the endoscope probe 30, a gastroscope GDB-VO-G-10, manufactured by LOMO Inc., is used. To form the multiple light source 10, a mercury arc lamp, DRSH-250-2, along with an optical filter (not shown in FIG. 1), which lets only the light with 380 nm-580 nm wavelength to pass through, are used as an excitation light source for irradiation, and a halogen lamp of KGM9-75 model is used for illumination light lighting. Such multiple light source 10 provides the excitation light of a short wavelength, 380 nm-580 nm, of a large output (output of more than 150 mW from the distal end of an endoscope when using the contrast agent, 5-amino-levulinic acid, ALA) during fluorescence endoscopy process. In the multiple light source 10, lighting condition can be changed by operating the light path switch 105 and the light source switch 104, which are installed either separately or together.

As for the color CCD camera 60, a commercial single matrix color CCD micro camera, GP-KS163 of Panasonic, Medical & Industrial Video Company, is used, and for the monochromatic CCD camera 70, a specialized high sensitive monochromatic CCD camera that belongs to TVIST, the CCD measuring system, is used. At wavelength of 550 nm, a sensitivity threshold value of the TVIST system is at 8×10⁻⁸W/m² in one second exposure time.

The CCD camera operates according to the signal charging principle, which in turn provides higher definition and wider dynamic range compare to a camera that uses an image intensifier. In addition, the CCD camera is small in size, light weighted, cheap and very reliable. When analyzing the fluorescence light, which is produced while using a medical agent, ALA, a 3 mm thick color glass, SZS-22, is used as an excitation light filter (not shown in FIG. 1) placed in between the multiple light source 10 and the optical cable 20, and a 2 mm thick color glass, KS-13, is used as the light shielding filter 103. Dichroic mirror 50 has characteristics of being a good reflector for light of wavelength greater than 580 nm and permeating light of wavelength shorter than 580 nm well. While performing a diagnosis under the illumination light, the dichroic mirror 50 is not to be included in the light path, by controlling the light path switch 105.

The light path switch 105, which adjusts the location of the dichroic mirror 50, is to be controlled in connection with the light source switch 104. An IBM compatible PC, equipped with a Pentium III-750 MHZ microprocessor, 128 Mb of RAM, 13.5 Gbytes hard-disk drive and a 17-inch monitor, is used as the controller 80. A video processing board uses a program, supplied exclusively from DC-30+, System TVIST Frame Grabber and Dual Video, in order to control the CCD devices and input/output from other equipments, store images and video films, process and analyze CCD images.

Subsequently, operations of the apparatus of the present invention, constructed as illustrated in FIG. 1, is to be described along with a description of a method of the invention, which is applied to the same.

The apparatus of the present invention operates under two conditions: an illumination light condition and a fluorescence light condition. Switching from one condition to another is done by operating the light path switch 105 that switches the light path by folding or unfolding the dichroic mirror 50. The dichroic mirror 50 gets included in the light path when checking the fluorescence light condition, and gets excluded when checking the illumination light condition. By operating the light path switch 50 and the light source switch 104 simultaneously; lighting of the multiple light source 10 can be selected to be either the fluorescence light or the illumination light.

At first, operation of the present invention under the fluorescence light condition is to be explained.

Light of the spectrum range that is selected by the light source switch 104; in other words, the excitation light of a short wavelength for the fluorescence light condition is to be reached at the diagnostic region 1 via the optical fiber bundle 31, which is used as the light transmitting path of the endoscope 30, after going through the optical cable 20. The excitation light of 380 nm-580 nm wavelength excites the diagnostic region 1, and by the contrast agent, ALA, fluorescence light, that has a wide wavelength of greater than 600 nm, is to be generated at an abnormal tissue site, such as a tumor, and at the same time, the reflective light of the excitation light of short wavelength, 380 nm-580 nm, is to be generated at the diagnostic region 1 except the abnormal tissue site.

The fluorescence light and the excitation light are to be transmitted into the dichroic mirror 50 by going through the optical fiber bundle 32, an image transmission path, then through the ocular lens 40. Among the light transmitted into the dichroic mirror 50, the reflective light of the excitation light gets passed through the dichroic mirror 50, whereas the fluorescence light gets divided into separate paths after reflected off from the same.

The reflective light gets inputted into the color CCD camera 60 through the objective lens 101. The color CCD camera 60, then, produces color images according to the inputted reflective light; and at the same, the fluorescence light gets inputted into the high sensitive monochromatic CCD camera 70 through the objective lens 102. The monochromatic CCD camera 70, then, produces high sensitive monochromatic images according to the inputted fluorescence light. Here, the monochromatic images form high sensitive images of abnormal tissue sites, such as a cancer site, whereas the color images form background images of the abnormal tissue sites. In other words, the color images, that are to be shown by the color CCD camera 60 based on the reflective light of the excitation light, are background images that are used to grasp the location of the irradiated tissue sites and to trace the change in location of the tissue site of an active organ. Moreover, the color images are used to adjust locations of the distal end of the endoscope probe and the surface of an organ. Whereas the monochromatic images of the monochromatic CCD camera 70 are to be used for discovering a malignant tumor according to the change in fluorescence light intensity at the location of tumor cells.

The color images and the monochromatic images, generated from the color CCD camera 60 and the monochromatic CCD camera 70, are provided as the inputs into the controller 80. The controller 80 processes the inputted monochromatic images and the color images into digital data; stores and analyzes the data; and displays the monochromatic images and the color images on the screen of the displaying device 90. In the sample application, the controller 80 is either to show the monochromatic images and the color images simultaneously on the screen in real-time, or to display the synthesized images on the screen of the displaying device 90, where the monochromatic image and the color image are synthesized into a single image.

Although the description of the operation of the apparatus of the present invention under the fluorescence light conditions has been made, the additional description for clarifying the technical characteristic of the present invention is as follows.

The spectrum range of the excitation light and the characteristic of the light shielding filter depend on the light spectral property of a fluorescence substance. When using the contrast agent, ALA, protoporphyrin IX is the fluorescence substance. The spectrum range of the excitation light of such contrast agent is 380 nm-580 nm, and blue-green light (or purple-green), which has the optical spectrum range of such wave length, is used as the light source for the fluorescence light conditions of the multiple light source 10.

Use of the designated light of wide optical spectrum range (in other words, excitation light of 380 nm-580 nm) that includes wide visible light spectrum portion, allows a doctor, as a user, to determine the best direction to look for in the interior of a human organ by increasing the excitation light intensity and providing sufficient background image data.

In order to make an objective evaluation of the fluorescence light intensity, the controller 80 is to perform a detailed image intensity calculation, an embodiment of an image onto a monitor screen, and storing the calculated intensity along with the image in real-time in accordance with a relative brightness (e.g. brightest part) selection. Moreover, analysis of the obtained fluorescence image is to be performed by the controller 80.

The brightness of a fluorescence image is not only affected by the substance of the diagnostic region under the examination, but also is affected by series of elements according to the endoscope apparatus and characteristics of the method. Change in distance between the distal end of the endoscope probe and the diagnostic region during the examination; lack of uniformity of light in the endoscope's field of view; brightness reduction of an image element, which is distributed on the outside of the optical axis of an objective lens, of an endoscope; and/or change in flux of the excitation light due to a lamp replacement or aging of a lamp are primary factors affecting the brightness of the fluorescence image. Such factors accidentally and systematically produce measuring errors, and make comparisons between the results, which are collected at different time periods and by the different instruments, difficult.

In order to reduce errors caused by change in distance to the tissue site, the reference distance can be maintained by pushing the instrument in through the endoscope instrument channel. And to reduce errors caused by change in sensitivity of the apparatus according to the lighting exchange and the field of view, normalization of fluorescence images of the tissue site, based on the collected data from the correction measuring process using the reference test sample 200, is to be achieved.

In other words, by processing series of movements under fluorescence light condition of the present invention using the reference test sample 200 that has an optical characteristic same/similar to that of a normal object, as described above, the normalization of the fluorescence images of the tissue site, based on the stored standard fluorescence image related data, is to be achieved when performing an actual endoscopy. The normalization is to be achieved after storing the standard fluorescence images and the related data (e.g. brightness of fluorescence light, analysis data of fluorescence image) collected from the controller 80.

By achieving the normalization of the fluorescence images of the tissue site, dependencies, caused by the distance from the distal end 30a of the endoscope probe 20 to the tissue site 1, and different light-collection effects of the objective lens 33 according to unevenness of lighting on the tissue site 1 and the field of view of the endoscope probe 30, are neglected when making fluorescence images of the tissue site 1.

Periodical adjustment of the apparatus of the present invention according to the reference test sample 200, promotes accuracy of the examination by removing the time dependent fluctuation effect of characteristic of the instrument when changing the lighting device and replacing parts.

Description of the operation of the present invention under illumination light condition is as follows.

When observing with illumination light, the dichroic mirror 50 is to be removed from the path of optical ray by operating the light path switch 105. At the same time, the light source switch 104 operates the multiple light source 10 with the illumination light condition. The illumination light, reflected from the tissue site 1, goes directly into the color CCD camera 60 without any disturbance from the dichroic mirror 50, after passing through the optical fiber bundle 32 and the ocular lens 40 of the endoscope probe 30. Furthermore, the illumination light forms the standard color images via the controller 80 and the displaying device 90.

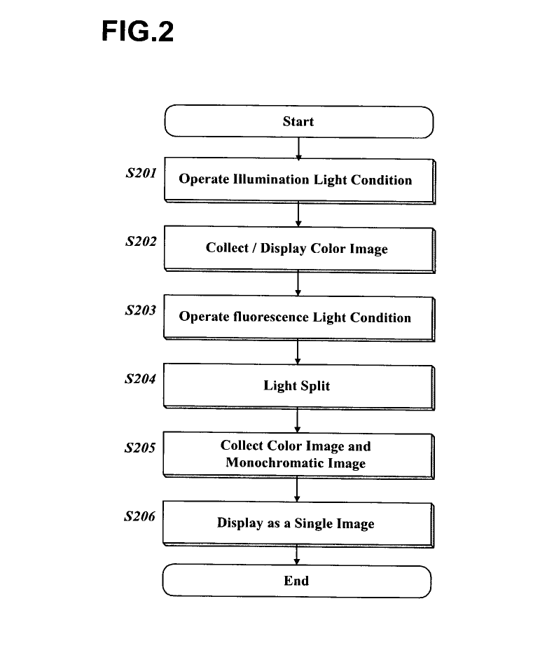

The description of the operation of the fluorescence endoscope apparatus in accordance with the exemplary embodiment of the present invention has been made along with the description of the method for imaging tissue within a body using the apparatus of the present invention. The description of the technical characteristics of the method for imaging tissue within a body, according to the exemplary embodiment of the present invention, will now be described with reference to FIG. 2.

FIG. 2 is a flowchart illustrating a method for imaging tissue within a body in accordance with an exemplary embodiment of the apparatus of FIG. 1 of the present invention.

At first, start the operation after manipulate and set the light source switch 104 and the light path switch 105 to take the illumination light condition. If illuminating S201 the tissue site 1 that has the fluorescence substance (e.g. contrast agent, ALA) by the light source of the illumination light placed in the multiple light source 10, then the corresponding reflective light of the illumination light gets inputted directly into the color CCD camera 60 to produce color images without passing through the dichroic mirror 50 that is folded to be free from the light path. These color images get processed, analyzed, and stored after inputted into the controller 80, and at the same time, the images of the diagnostic region are to be displayed on the displaying device 90 to allow a user to make an observation S202.

When the observation region is seen as a morphologically abnormal structure or as an abnormal color that provides suspicion of an existence of a tumor, it is to be operated after manipulating the light source switch 104 and the light path switch 105 to move on to the fluorescence testing process. If illuminating S203 the tissue site 1 that has the fluorescence substance (e.g. contrast agent, ALA) by the light source of the excitation light, that has wide spectrum range of short wavelength (380 nm-580 nm), placed in the multiple light source 10, then the fluorescence light, generated at the tumor site by the contrast agent, ALA and the corresponding reflected excitation light (380 nm-580 nm wavelength) get projected into the dichroic mirror 50 located in the path of the optical ray, and then the path of each optical ray gets divided. The reflected excitation light, then, gets inputted into the color CCD camera 60 after passing through the dichroic mirror 50, and at the same, the fluorescence light gets inputted into the monochromatic CCD camera 70 after reflected by the dichroic mirror 50.

The color CCD camera 60 collects color images, which become the background of the diagnostic region, based on the inputted reflected excitation light that has wide spectrum range (380 nm-580 nm), and at the same time, the monochromatic CCD camera 70 collects the monochromatic images S205, which display a tumor at the diagnostic region, based on the inputted fluorescence light.

The controller 80 digitally processes the collected color background images and the monochromatic tumor images; stores and analyzes the data; displays the monochromatic images and the color images simultaneously on the screen of the displaying device 90, or displays the synthesized images on the screen of the displaying device 90, after synthesizing the monochromatic image and the color image into a single image S206.

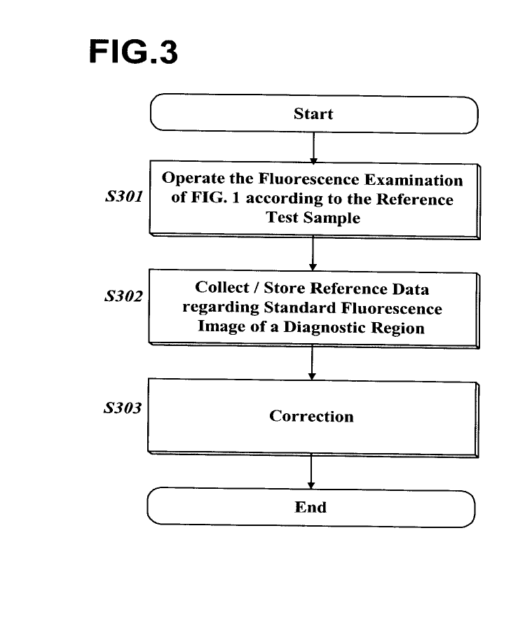

On the other hand, before begin the examination as described above, the reference data regarding the standard fluorescence images of the tissue site of the examining object is to be collected by executing the operation of the fluorescence testing processes (S203-S205) methodically with the reference test sample 200 as illustrated in FIG. 3 S201. Then the collected data gets stored into the controller 80, S302, and the fluorescence images, collected during the examination of the actual object through correcting the apparatus of the present invention based on the reference data S303, are to be corrected.

A technical object with a surface that has optically similar characteristics when compare to the stomach area, as the actual examining object, is to be selected as the reference test sample 200. The correction process S303 records and stores the reference data into the controller 80 after making the fluorescence images of the reference test sample 200 as the reference data at a fixed distance (for instance, 10 mm) apart from the diagnostic region that corresponds with the distance in a general endoscopy.

According to the following methods, the stored reference data is to be used when the correction process S303 is being operated. The signal that corresponds to the brightest spots of the reference test sample 200 screen is used for correcting the numerical value of photometric parameter of the fluorescence images of the diagnostic region 1. Moreover, change in image signals of the reference test sample 200 at various points of the endoscope's field of view is used to correct unevenness of the fluorescence light signal distribution at the diagnostic region 1.

More detailed description about the endoscopy, that uses the reference test sample 200 of the step S301 as the object, is as follows.

The illumination light source of the multiple light source 10 is used to illuminate the diagnostic region 1 that has the fluorescence substance, and color images at various regions of an organ are collected via the color CCD camera 60 to be observed through the displaying device 90 where the dichroic mirror 50 is not folded. In the case where abnormality of a morphological structure or a region that has a relatively suspicious color is observed, the multiple light source 10 is to be set to provide the short wavelength examination fluorescence light of a large output power (in other words, excitation light), and coverts to the optical system, comprised of the high sensitive monochromatic CCD camera 70, to perform the fluorescence testing process. Digitally stored irregular fluorescence light signal is to be removed through the image process at the controller 80. The fluorescence images are to be observed from the displaying device 90. In this experiment, special colored paper paint is used on the surface of the reference test sample 200, and the fluorescence and the reflective properties of the paper paint are same/similar with those of stomach mucous surface of a patient who took the contrast agent, ALA. After applying the reference test sample measuring system during a sample experiment, an accuracy enhancement and high reproducibility in correction of the fluorescence images were observed, and has provided application reliability.

As described above in detail, in accordance with the fluorescence endoscope apparatus and the method for imaging tissue within a body using the same, following effects can be produced.

1) By the excitation light and the illumination light of the illuminator, a fluorescence examination as well as a general endoscopy can be performed.

2) When switching from the fluorescence examination to an illumination light observation, the endoscope does not need to be detached from the CCD module, and the switching process is to be performed efficiently by simply changing the location of the dichroic mirror.

3) The two CCD camera systems connected to the control unit can display the fluorescence light images and the reflective light images simultaneously on the displaying unit in real-time, from the diagnostic region.

4) Brightness of the fluorescence images is to be enhanced by reducing the extra fluorescence light, which is generated by the excitation light, using the dichroic mirror.

5) If non-coherent light source rather than laser is used to excite the fluorescence light, then the use of various indicators for tumors is allowed without changing the lighting device. Non-coherent fluorescence light source is generally very cheap, reliable and simple compare to the laser light source. In the lighting device of such kind, selecting the most suitable parts can provide the excitation light of a large output power and possibility of lighting with light of wide wavelength range (visible spectrum range of 380 nm-580 nm when processed with ALA, the contrast agent), which allows a doctor to evaluate the examined region of an organ with reliability, and allows to control the location of the distal end of an endoscope.

6) Under the fluorescence light condition, transmission of the fluorescence light that corresponds with the excitation light and the reflective light is to be performed by using the two different CCD cameras, and each of defined functions is to be optimally performed. In other words, the high definition color background images are provided in the first case, and the high sensitive monochromatic images are provided as pictures of an abnormal region in the second case.

7) The histogram, that shows signal distribution of the screen through computer analysis done by the controller, allows quantitative evaluation of the fluorescence light intensity of an image of a diagnostic region. 8) Operation of correcting instrument in accordance with the reference test sample, computer image processing of the fluorescence image, and use of distance fixing method are improving the accuracy of the fluorescence analysis by reducing errors, which are caused by change in distance between the distal end of an endoscope probe and the surface of the object; unevenness of the lighting; different light-collection in accordance with the field of view of an endoscope; and change in sensitivity of an instrument in accordance with time and location. 9) Along with data operation, use of computer multimedia unit allows an observation result to be stored as digital video clip.

As a result, all the advantages of the present invention, as I) described above, allow more accurate examination to be performed, and provide convenient use of the instrument.

What is claimed is:

1. A fluorescence endoscope apparatus for imaging tissue within a body that has a characteristic of comprising;
a multiple light source unit, equipped with multiple light sources of various wavelengths, for providing a selected light;
a light transmission unit, where an exit path for transmitting and radiating the provided light and an incidence path for transmitting the incidence light in correspondence with said radiation are formed in parallel with each other, and an objective lens is installed on said incidence path, to be able to be inserted in to an interior of a body;
a light splitting unit for separating light transmitted via said incidence path, into primary and secondary light by letting the light to pass through or to reflect according to the light type;
a primary image processing unit for collecting primary images in accordance with the primary light that has passed through;
a secondary image processing unit for collecting secondary images in accordance with the reflected secondary light;
a control unit for processing, analyzing, storing and synthesizing the collected primary and secondary images; and
a display unit for displaying said primary and secondary images or synthesized images that are processed in said control unit.

2. The fluorescence endoscope apparatus of claim 1, wherein said multiple light source unit is equipped with illumination light and excitation light as the light sources.

3. The fluorescence endoscope apparatus of claim 2, wherein the light sources of said illumination light and said excitation light are organized with two different lamps.

4. The fluorescence endoscope apparatus of claim 3, wherein the lamp for said illumination light is a halogen lamp and the lamp for said excitation light is a mercury lamp.

5. The fluorescence endoscope apparatus of claim 2, wherein said light sources of the illumination light and the excitation light are organized as a single combined lamp.

6. The fluorescence endoscope apparatus of claim 5, wherein said combined lamp is a xenon lamp.

7. The fluorescence endoscope apparatus of claim 1, wherein said light splitting unit is formed as a dichroic mirror that allows reflected excitation light to pass through and fluorescence light from said excitation light to be reflected.

8. The fluorescence endoscope apparatus of claim 7, wherein said dichroic mirror is installed in a way to selectively escape from said light path for passing and reflecting light.

9. The fluorescence light endoscope apparatus of claim 2, wherein said excitation light has a spectrum range of 380 nm-580 nm .

10. The fluorescence light endoscope apparatus of claim 7, wherein said fluorescence light has a wavelength of greater than 580 nm.

11. The fluorescence light endoscope apparatus of claim 1, wherein said primary image processing unit collects color images, and said secondary image processing unit collects high sensitive monochromatic images.

12. The fluorescence light endoscope apparatus of claim 1, wherein objective lenses are respectively installed on incidence paths of said primary and said secondary image processing unit.

13. The fluorescence light endoscope apparatus of claim 1, wherein a light shielding filter, which permeates only the fluorescence light, is installed on the incidence path of said secondary image processing unit.

14. The fluorescence endoscope apparatus of claim 13, wherein said light shielding filter absorbs light of wavelength less than 600 nm.

15. A fluorescence endoscope apparatus of claim 1, wherein said control unit stores data regarding standard images of corresponding object under examination as reference images, then corrects the collected secondary images from the actual object under examination according to the reference images.

16. The fluorescence endoscope apparatus of claim 15, wherein said reference images are collected via said secondary images from the reference test sample of a model that has same/similar optical characteristics as those of the actual object under examination.

17. A method for imaging tissue within a body using the fluorescence endoscope apparatus, comprising the steps of:
collecting reference data regarding standard fluorescence images of corresponding object under examination from a reference test sample of a model that has same/similar optical characteristics as an actual object under examination;
illuminating a diagnostic region of said object under examination using the illumination light;
collecting and displaying color images based on the reflective light reflected by lighting of said illumination light;
lighting said diagnostic region using the excitation light that has a wide spectrum range;
collecting high sensitive monochromatic images based on the fluorescence light from the lighting of said excitation light, and at the same time, collecting color images based on the reflective light from the same;
correcting said collected high sensitive monochromatic images and brightness of said fluorescence light, based on said collected reference data; and
displaying said corrected high sensitive monochromatic image and color image on the screen simultaneously, or displaying as a single synthesized image after synthesis.

18. The method of claim 17, wherein said correcting step corrects:
a numerical value of a photometric parameter of said monochromatic image as a fluorescence light image of said object under examination, based on signals that are in correspondence with the brightest spots of the displayed image obtained from said reference test sample in said collecting step; and
unevenness of fluorescence light distribution on said object under examination, based on changes of image signals of said reference test sample at various spots of the field of view of the endoscope.

19. The method of claim 17, wherein said collected monochromatic images and color images are stored as digital video clips, then mutually synthesized.

20. The method of claim 17, wherein fluorescence light intensity of said monochromatic images are calculated through a histogram analysis of relative distribution of image signal regarding the displaying screen.

21. The method of claim 20, wherein said calculated fluorescence light intensity data is displayed as digital numbers on the screen along with the images.
